(19) United States
(12) Patent Application Publication
(10) Pub. No.: US 2023/0018429 A1
(43) Pub. Date: Jan. 19, 2023

(54) PROCESS FOR SYNTHESIS OF (3-CHLORO-2-PYRIDYL)HYDRAZINE

(71) Applicants: FMC CORPORATION, PHILADELPHIA, PA (US); FMC AGRO SINGAPORE PTE. LTD., SINGAPORE (SG)

(72) Inventors: DEVARAJAN CHOCKALINGAM, GUJARAT (IN); VIPUL DUDHAT, GUJARAT (IN); RAJU MAHADEV KHARATKAR, GUJARAT (IN); JIANHUA MAO, SHANGHAI (CN); PANKAJKUMAR VEKARIYA, GUJARAT (IN); DHARMESH BALVANTRAI BHATT, GUJARAT (IN)

(21) Appl. No.: 17/778,472

(22) PCT Filed: Nov. 22, 2020
(86) PCT No.: PCT/US20/61709

§ 371 Date: May 20, 2022

Related U.S. Application Data
(60) Provisional application No. 62/939,119, filed on Nov. 22, 2019.

(51) Int. Cl. C07D 213/61; B01J 27/24; B01J 23/00

(52) U.S. Cl. CPC C07D 213/61; B01J 27/24; B01J 23/007

(57) ABSTRACT

Described herein are novel methods of synthesizing (3-chloro-2-pyridyl)hydrazine. Compounds prepared by the methods disclosed herein are useful for preparation of certain anthranilamide compounds that are of interest as insecticides, such as, for example, the insecticides chlorantraniliprole and cyantraniliprole.

### CROSS-REFERENCE TO RELATED APPLICATION

This application claims the benefit of U.S. Provisional Application No. 62/939,119 filed Nov. 22, 2019.

### FIELD OF INVENTION

This disclosure is directed to novel methods of synthesizing (3-chloro-2-pyridyl)hydrazine. Compounds prepared by the methods disclosed herein are useful for preparation of certain anthranilamide compounds that are of interest as insecticides, such as, for example, the insecticides chlorantraniliprole and cyantraniliprole.

### BACKGROUND

Conventional processes for the production of (3-chloro-2-pyridyl)hydrazine are subject to several industrial concerns, such as hazardous materials, high cost, relatively long method steps, and complicated operations. The use of costly and difficult-to-recover reagents and organic solvents is undesirable.

The present disclosure provides novel methods useful for preparing 5-Bromo-2-(3-chloropyridin-2-yl)-2H-pyrazole-3-carboxylic acid and derivatives thereof. The benefits of the methods of the present disclosure compared to previous methods are numerous and include reduced cost, eliminated need for mixed solvent separations, reduced waste, relatively short method steps, simplified operation complexity, and reduced process hazards.

### BRIEF DESCRIPTION

In one aspect, provided herein is a method of preparing a compound of Formula II, wherein

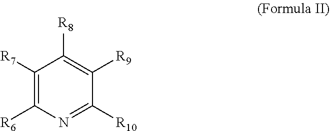

each of R₆-R₁₀ is independently selected from hydrogen, halogen, and hydrazino; wherein at least one of R₆-R₁₀ is hydrazino, the method comprising

I) forming a mixture comprising
A) a compound of Formula I, wherein

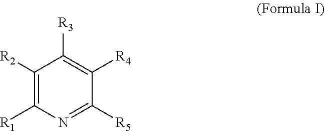

each of R₁-R₅ is independently selected from hydrogen and halogen
wherein at least one of R₁-R₅ is halogen;
B) an inorganic hydrazine derivative;
C) a catalyst;
D) an inorganic base or a salt; and
E) optionally an organic solvent optionally comprising water; and

II) reacting the mixture.

### DETAILED DESCRIPTION OF THE DISCLOSURE

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “halogen”, either alone or in compound words such as “haloalkyl”, includes fluorine, chlorine, bromine or iodine. Further, when used in compound words such as “haloalkyl”, said alkyl may be partially or fully substituted with halogen atoms which may be the same or different.

When a group contains a substituent which can be hydrogen, for example R⁴, then, when this substituent is taken as hydrogen, it is recognized that this is equivalent to said group being unsubstituted.

The term “hydrazino” includes, without limitation, a functional group comprising a hydrazino bond (—HN—NH2).

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

The embodiments of this disclosure include:

Embodiment 1. A method of preparing a compound of Formula II, wherein

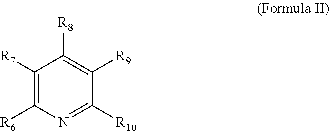

each of R₆-R₁₀ is independently selected from hydrogen, halogen, and hydrazino; wherein at least one of R₆-R₁₀ is hydrazino, the method comprising

I) forming a mixture comprising
A) a compound of Formula I, wherein

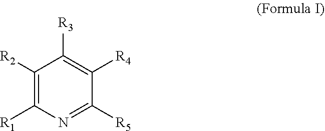

each of R₁-R₅ is independently selected from hydrogen and halogen;
wherein at least one of R₁-R₅ is halogen;
B) an inorganic hydrazine derivative;
C) a catalyst;
D) an inorganic base or a salt; and
E) optionally an organic solvent optionally comprising water; and

II) reacting the mixture.

Embodiment 2. The method of embodiment 1, wherein the mixture further comprises an aqueous solvent comprising water.

Embodiment 3. The method of embodiment 2, wherein the aqueous solvent is water.

Embodiment 4. The method of embodiment 1, wherein the inorganic hydrazine derivative is selected from aqueous hydrazine, hydrazine hydrates, hydrazine salts, and combinations thereof.

Embodiment 5. The method of embodiment 4, wherein the inorganic hydrazine derivative is aqueous hydrazine comprising hydrazine monohydrate.

Embodiment 6. The method of embodiment 1, wherein the inorganic hydrazine derivative is present in an amount above 1 m/m, above, 2 m/m, above 3 m/m, above 5 m/m or above 10 m/m; or from about from about 1 m/m to about 10 m/m, or from about 1 m/m/ to about 3 m/m, from about 2 m/m/ to about 5 m/m, or from about 3 m/m/ to about 10 m/m.

Embodiment 7. The method of embodiment 1, wherein the catalyst is selected from phase transfer catalysts, organic base catalysts, and combinations thereof.

Embodiment 8. The method of embodiment 1, wherein the catalyst is selected from quaternary ammonium salts, crown ethers, inorganic salts, organic bases, and combinations thereof.

Embodiment 9. The method of embodiment 7, wherein the phase transfer catalyst is selected from tetra butyl ammonium chloride, tetra butyl ammonium bromide, aliquat-336, 18-crown-6, benzyltriethylammonium chloride, and combinations thereof.

Embodiment 10. The method of embodiment 9, wherein the phase transfer catalyst is aliquat-336.

Embodiment 11. The method of embodiment 7, wherein the organic base catalyst is selected from 1,4-diazabicyclo[2.2.2]octane, 1,8-Diazabicyclo[5.4.0]undec-7-ene, and combinations thereof.

Embodiment 12. The method of embodiment 1, wherein the inorganic base is selected from sodium hydroxide, potassium hydroxide, sodium carbonate, potassium carbonate, powdered potassium carbonate (400 mesh), potassium bicarbonate, sodium bicarbonate, dipotassium phosphate, tripotassium phosphate, disodium phosphate, trisodium phosphate, monosodium sodium methoxide, potassium t-butoxide, and combinations thereof.

Embodiment 13. The method of embodiment 12, wherein the inorganic base is potassium carbonate or tripotassium phosphate.

Embodiment 14. The method of embodiment 1, wherein the salt is selected from sodium sulfate, potassium sulfate, sodium chloride, potassium chloride, potassium iodide, and combinations thereof.

Embodiment 15. The method of embodiment 1, wherein the organic solvent is selected from methanol, ethanol, propanol, isopropanol, n-butanol, t-butanol, heptane, toluene, n-octane, and combinations thereof.

Embodiment 16. The method of embodiment 1, wherein the mixture is substantially free of organic solvents.

Embodiment 17. The method of embodiment 1, wherein the compound of Formula I is selected from 2-chloropyridine, 2,3-dichloropyridine, 2,6-dichloropyridine, and combinations thereof.

Embodiment 18. The method of embodiment 1, wherein the compound of Formula II is selected from 2-hydrazinopyridine, (3-chloro-2-pyridyl)hydrazine, (6-chloro-2-pyridyl)hydrazine, and combinations thereof.

Embodiment 19. The method of embodiment 1, wherein the method step of reacting the mixture occurs at a temperature in the range of about 90° C. to about 115° C.

Embodiment 20. The method of embodiment 1, wherein the method step of reacting the mixture occurs during a reaction time in the range of about 6 hours to about 30 hours.

Embodiment 21. The method of embodiment 1, wherein the method step of reacting the mixture occurs at a pressure in the range of about 1.0332 kg/cm² to about 5 kg/cm².

In one aspect, a compound of Formula II is prepared according to a method represented by Scheme 1. The R groups are as defined anywhere in this disclosure.

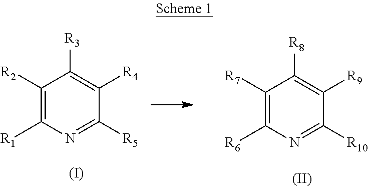

This aspect includes mixing a compound of Formula I, an inorganic hydrazine derivative, a catalyst, a base or a salt, and optionally a solvent, and reacting the mixture.

In one embodiment, the mixture further comprises an aqueous solvent comprising water. In another embodiment, the mixture further comprises water.

In one embodiment, the inorganic hydrazine derivative is selected from aqueous hydrazine, hydrazine hydrates, hydrazine salts, and combinations thereof. In one embodiment the aqueous hydrazine comprises hydrazine monohydrate. In one embodiment, the inorganic hydrazine derivative is an aqueous hydrazine solution with a concentration in the range of about 30% to about 64%. In one embodiment, the inorganic hydrazine derivative is present in the mixture in an amount above 1 m/m, above, 2 m/m, above 3 m/m, above 5 m/m or above 10 m/m; or from about from about 1 m/m to about 10 m/m, or from about 1 m/m/ to about 3 m/m, from about 2 m/m/ to about 5 m/m, or from about 3 m/m/ to about 10 m/m.

In one embodiment, the catalyst is selected from phase transfer catalysts, organic base catalysts, and combinations thereof. In another embodiment, the catalyst is a phase transfer catalyst selected from tetra butyl ammonium chloride, tetra butyl ammonium bromide, aliquat-336, 18-crown-6, benzyltriethylammonium chloride, and combinations thereof. In another embodiment, the catalyst is an organic base catalyst selected from 1,4-diazabicyclo[2.2.2]octane (DABCO), 1,8-Diazabicyclo[5.4.0]undec-7-ene (DBU), and combinations thereof. In one embodiment, the catalyst is present in the mixture in an amount from about 0.1 mol % to about 1 mol %. In another embodiment, the catalyst is present in the mixture in an amount from about 0.25 mol % to about 0.75 mol %.

In one embodiment, the base is selected from inorganic base and organic base. In another embodiment, the base is an inorganic base selected from sodium hydroxide, potassium hydroxide, sodium carbonate, potassium carbonate, powdered potassium carbonate (400 mesh), potassium bicarbonate, sodium bicarbonate, dipotassium phosphate, tripotassium phosphate, disodium phosphate, trisodium phosphate, monosodium sodium methoxide, potassium t-butoxide, and combinations thereof. In one embodiment, the base is present in the mixture in an amount from about 0.1 mol % to about 2 mol %. In another embodiment, the base is present in the mixture in an amount from about 0.25 mol % to about 1.5 mol %.

In one embodiment, the salt is an inorganic salt. In another embodiment, the salt is selected from sodium sulfate, potassium sulfate, sodium chloride, potassium chloride, potassium iodide, and combinations thereof.

In one embodiment, the solvent is selected from organic solvent, aqueous solvent, and combinations thereof. In one embodiment the aqueous solvent comprises water. In another embodiment, the solvent is an organic solvent is selected from methanol, ethanol, propanol, isopropanol, n-butanol, t-butanol, heptane, toluene, n-octane, and combinations thereof. In another embodiment, the mixture is substantially free of organic solvent. In another embodiment, the no organic solvent is present in the mixture.

In one embodiment, the compound of Formula I is selected from 2-chloropyridine, 2,3-dichloropyridine, 2,6-dichloropyridine, and combinations thereof. In another embodiment, the compound of Formula I is selected from 2-bromopyridine, 2,3-dibromopyridine, 2,6-dibromopyridine, and combinations thereof.

In one embodiment, the compound of Formula II is selected from 2-hydrazinopyridine, (3-chloro-2-pyridyl)hydrazine, (6-chloro-2-pyridyl)hydrazine, and combinations thereof.

In one embodiment, the method step of reacting the mixture occurs at a temperature in the range of about 50° C. to about 200° C. In another embodiment, the method step of reacting the mixture occurs at a temperature in the range of about 90° C. to about 115° C.

In one embodiment, the method step of reacting the mixture occurs during a reaction time in the range of about 6 hours to about 30 hours.

In one embodiment, the method step of reacting the mixture occurs at a pressure in the range of about 1.0332 kg/cm² to about 10 kg/cm². In another embodiment, the method step of reacting the mixture occurs at a pressure in the range of about 1.0332 kg/cm² to about 5 kg/cm².

In one embodiment, when this aspect includes a mixture comprising an inorganic base and a phase transfer catalyst, the necessary amount of hydrazine is substantially decreased.

In one aspect, (3-chloro-2-pyridyl)hydrazine is prepared according to a method represented by Scheme 2.

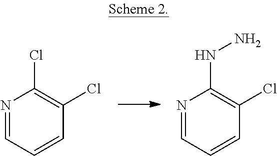 [SMILES: Clc1cccnc1Cl ; NNc1ncccc1Cl]

In one aspect, 2-Hydrazinopyridine is prepared according to a method represented by Scheme 3.

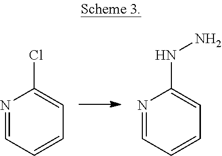 [SMILES: Clc1ccccn1 ; NNc1ccccn1]

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Example 1. No Organic Solvent

243.2 g (2.4 m/m) of hydrazine mono hydrate (100%) was added to a mixture of 300.0 g (2.027 moles) of 2,3-Dichloropyridine, 2.1 g of Aliquat 336 (0.25 mole %, 0.7% wrt to 2,3-Dichloropyridine), and 98 g of K₂CO₃ (0.35 m/m) in a single lot at 25-30° C. No exotherm was observed. For reference, 100% hydrazine mono hydrate is equivalent to 64% aqueous hydrazine. The mixture was heated to 110-115° C. in 1 hour and held at reflux for 14 hours. LC A % monitoring shows 99.2% (3-chloro-2-pyridyl)hydrazine and 0.1% of 2,3-Dichloropyridine. The reaction mass was cooled to 80° C., 300 g water was added, and the reaction mass was cooled further to 25-30° C. under stirring. The precipitate formed was collected by filtration and the mass was washed with 1000 g water and dried at 60° C. under 100-150 torr vacuum for 12 hours until constant weight was obtained.

TABLE 1
Output summary.

| | |
|---|---|
| Total weight of (3-chloro-2-pyridyl)hydrazine | 280 g |
| Nature of product | Light brownish |
| | crystalline powder |
| Purity | 96% |
| Yield | 92.4% |
| Melting point | 163.5-164.9° C. |

### Example 2. N-Butanol as a Solvent

20.3 g (2.4 m/m) of 100% hydrazine mono hydrate was added to a mixture of 25.0 g (0.169 moles) of 2,3-Dichloropyridine, 0.35 g of Aliquat 336 (0.5 mole %, 1.4% wrt to 2,3-Dichloropyridine), 88 g of n-butanol, and 16.3 g of K₂CO₃ (0.7 m/m) in a single lot at 25-30° C. No exotherm was observed. For reference, 100% hydrazine mono hydrate is equivalent to 64% aqueous hydrazine. The mixture was heated to 102-105° C. in 1 hour and held at reflux for 30 hours. LC A % monitoring shows 90.6% (3-chloro-2-pyridyl)hydrazine and 8.09% of 2,3-Dichloropyridine. The reaction mass was cooled to 80° C., 40 g water was added, and the reaction mass was cooled further to 25-30° C. under stirring. The precipitate formed was collected by filtration and the mass was washed with 150 g water and dried at 60° C. under 100-150 torr vacuum for 12 hours until constant weight was obtained.

TABLE 2
Output summary.

| | |
|---|---|
| Total weight of (3-chloro-2-pyridyl)hydrazine | 22.0 g |
| Nature of product | White crystalline |
| | powder |
| Purity | 99.76% (LC A %) |
| Yield | 90.5% (basis LC A %) |
| Melting point | 164.2-165.7° C. |

### Example 3. 2-Chloropyridine as a Reactant

26.42 g (2.4 m/m) of 64% aqueous hydrazine was added to a mixture of 25.0 g (0.22 moles) of 2-Chloropyridine, 0.35 g of Aliquat 336 (0.4 mole %, 1.4% wrt to 2-Chloropyridine), and 21.3 g of K₂CO₃ (0.7 m/m) in a single lot at 25-30° C. No exotherm was observed. For reference, 100% hydrazine mono hydrate is equivalent to 64% aqueous hydrazine. The mixture was heated to 102-105° C. in 1 hour and held at reflux for 21 hours. LC A % monitoring shows 59.45% 2-Hydrazinopyridine and 32.80% of 2-Chloropyridine. The reaction mass was cooled to 80° C., 40 g water was added, and the reaction mass was cooled further to 25-30° C. under stirring. Aqueous and organic layers were separated, and the organic layer was subjected to distillation at 60° C. under 100 torr vacuum for 10 hours until 2-Hydrazinopyridine (8 g, LCA %=93%) was obtained as residue. This crude product was further purified by crystallization with toluene to obtain crystalline 2-Hydrazinopyridine.

TABLE 3
Output summary.

| | |
|---|---|
| Total weight of 2-Hydrazinopyridine | 3.2 g |
| Nature of product | Brownish crystalline |
| | powder |
| Purity | 97.8% (LC A %) |
| Yield | 13% (basis LC A %) |
| Melting point | 40-43° C. |

### Example 4. Tripotassium Phosphate as Base

39.72 g (2.4 m/m) of hydrazine mono hydrate (100%) was added to a mixture of 50.0 g (0.331 moles) of 2,3-Dichloropyridine, 034 g of Aliquat 336 (0.25 mole %, 0.7% wrt o 2,3 Dichloropyridine) and 24.6 g of K₃PO₄ (0.35 m/m/) in a single lot at 25-30° C. Exotherm was observed up to 45° C. For reference, 100% hydrazine mono hydrate is equivalent to 64% aqueous hydrazine. The mixture was heated to 110-120° C. in 1 h and held at reflux for 5 hours. LC A % monitoring shows 98.5% (3-chloro-2-pyridyl)hydrazine and 0.1% of 2,3 Dichloropyridine. The reaction mass was cooled to 80° C., 50 g of water was added, and the reaction mass was cooled further to 25-30° C. under stirring. The precipitate formed was collected by filtration and the mass was washed with 165 g water and dried at 60° C. under 100-150 torr vacuum for 12 hours until constant weight was obtained.

TABLE 4
Output summary.

| | |
|---|---|
| Total weight of (3-chloro-2-pyridyl)hydrazine | 44.4 g |
| Nature of product | Light brownish |
| | crystalline powder |
| Purity | 99.36% (LC A %) |
| Yield | 92.8% (basis LC A %) |
| Melting point | 162.9-165.4° C. |

### Example 5. Potassium Hydroxide and Tripotassium Phosphate as Base

33.11 g (2 m/m) of hydrazine mono hydrate (100%) was added to a mixture of 50.0 g (0.331 moles) of 2,3-Dichloropyridine, 0.34 g of Aliquat 336 (0.25 mole %, 0.7% wrt to 2,3 Dichloropyridine), 7.03 g of K₃PO₄ (0.1 m/m) and 4.37 g of KOH (0.2 m/m) in a single lot at 25-30° C. Exotherm was observed up to 34-36° C. For reference, 100% hydrazine mono hydrate is equivalent to 64% aqueous hydrazine. The mixture was heated to 110-120° C. in 1 h. 17.48 g of KOH (0.8 m/m) was added in 4 equal lots at 2 hrs interval under heating and held at reflux for 2 hours after all 4 lots had been charged. LC A % monitoring showed 98.9% (3-chloro-2-pyridyl)hydrazine and 0.1% of 2,3-Dichloropyridine. The reaction mass was cooled to 80° C. and 50 g of water was added and the mass was further cooled to 25-30° C. under stirring. The precipitate formed was collected by filtration, and the mass was washed with 165 g water and dried at 60° C. under 100-150 torr vacuum for 12 h until constant weight was obtained.

TABLE 5
Output summary.

| | |
|---|---|
| Total weight of (3-chloro-2-pyridyl)hydrazine | 45.2 g |
| Nature of product | Light brownish |
| | crystalline powder |
| Purity | 98.46% (LC A %) |
| Yield | 93.6% (basis LC A %) |
| Melting point | 162.7-165.5° C. |

### Example 6. Lotwise Addition of Potassium Hydroxide as Base

18.8 g of 85% KOH (0.7 m/m) was added lot wise to a mixture of 60.0 g (0.405 moles) of 2,3-Dichloropyridine, 0.41 g of Aliquat 336 (0.25 mole %, wrt to 2,3 Dichloropyridine) and 48.6 g (2.4 m/m) of hydrazine mono hydrate (100%). The lot wise addition was performed by adding 20% (3.8 g, 0.14 m/m) of potassium hydroxide at hour 0 and the remaining potassium hydroxide (15 g, 0.56 m/m) at 8 equal lots at intervals of 2 hours each at 100° C. For reference, 100% hydrazine mono hydrate is equivalent to 64% aqueous hydrazine. The mixture was held at 110-115° C. for 16 h. LC A % monitoring shows 99.38% of (3-chloro-2-pyridyl)hydrazine and 0.635% of 2,3-Dichloropyridine. The reaction mass was cooled to 80° C. and 60 g of water was added and further cooled to 25-30° C. under stirring. The precipitate formed was collected by filtration, and the mass was washed with 200 g of water and dried at 60° C. under 100-150 torr vacuum for 12 h until constant weight was obtained.

TABLE 6
Output summary.

| | |
|---|---|
| Total weight of (3-chloro-2-pyridyl)hydrazine | 54.0 g |
| Nature of product | Light brownish |
| | crystalline powder |
| Purity | 97.7% (LC A %) |
| Yield | 92.5% (basis LC A %) |

### Example 7. Potassium Hydroxide as Base

18.8 g of KOH 85% (0.70 m/m) was added in a single lot at 25-30° C. to a mixture of 60.0 g (0.405 moles) of 2,3-Dichloropyridine, 0.41 g of Aliquat 336 (0.25 mole %, wrt to 2,3 Dichloropyridine) and 48.6 g (2.4 m/m) of hydrazine mono hydrate (100%). The resulting mixture was heated to 110-115° C. in 1 h and held at reflux for 17 hours. LC A % monitoring shows 99.4% (3-chloro-2-pyridyl)hydrazine and 2,3-Dichloropyridine-nil. The reaction mass was cooled to 80° C., 60 g of water was added and then further cooled to 25-30° C. under stirring. The precipitate formed was collected by filtration, the mass was washed with 200 g water and dried at 60° C. under 100-150 torr vacuum for 12 h until constant weight was obtained.

TABLE 7
Ouput summary.

| | |
|---|---|
| Total weight of (3-chloro-2-pyridyl)hydrazine | 46.0 g |
| Nature of product | Light brownish |
| | crystalline powder |
| Purity | 96.62% (LC A %) |
| Yield | 77.5% (basis LC A %) |

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of preparing a compound of Formula II, wherein 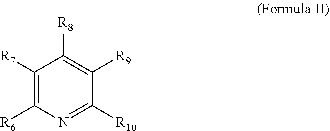
each of R₆-R₁₀ is independently selected from hydrogen, halogen, and hydrazino;
wherein at least one of R₆-R₁₀ is hydrazino, the method comprising
I) forming a mixture comprising
A) a compound of Formula I, wherein 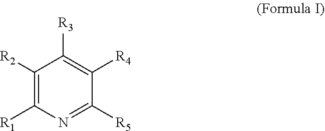
each of R₁-R₅ is independently selected from hydrogen and halogen
wherein at least one of R₁-R₅ is halogen;
B) an inorganic hydrazine derivative;
C) a catalyst;
D) an inorganic base or a salt; and
E) optionally an organic solvent optionally comprising water; and
II) reacting the mixture.

2. The method of claim 1, wherein the mixture further comprises an aqueous solvent comprising water.

3. The method of claim 2, wherein the aqueous solvent is water.

4. The method of claim 1, wherein the inorganic hydrazine derivative is selected from aqueous hydrazine, hydrazine hydrates, hydrazine salts, and combinations thereof.

5. The method of claim 4, wherein the inorganic hydrazine derivative is aqueous hydrazine comprising hydrazine monohydrate.

6. The method of claim 1, wherein the inorganic hydrazine derivative is present in an amount from about in an amount above 1 m/m.

7. The method of claim 1, wherein the catalyst is selected from phase transfer catalysts, organic base catalysts, and combinations thereof.

8. The method of claim 1, wherein the catalyst is selected from quaternary ammonium salts, crown ethers, inorganic salts, organic bases, and combinations thereof.

9. The method of claim 7, wherein the phase transfer catalyst is selected from tetra butyl ammonium chloride, tetra butyl ammonium bromide, aliquat-336, 18-crown-6, benzyltriethylammonium chloride, and combinations thereof.

10. The method of claim 9, wherein the phase transfer catalyst is aliquat-336.

11. The method of claim 7, wherein the organic base catalyst is selected from 1,4-diazabicyclo[2.2.2]octane, 1,8-Diazabicyclo[5.4.0]undec-7-ene, and combinations thereof.

12. The method of claim 1, wherein the inorganic base is selected from sodium hydroxide, potassium hydroxide, sodium carbonate, potassium carbonate, powdered potassium carbonate (400 mesh), potassium bicarbonate, sodium bicarbonate, dipotassium phosphate, tripotassium phosphate, disodium phosphate, trisodium phosphate, monosodium sodium methoxide, potassium t-butoxide, and combinations thereof.

13. The method of claim 12, wherein the inorganic base is potassium carbonate.

14. The method of claim 1, wherein the salt is selected from sodium sulfate, potassium sulfate, sodium chloride, potassium chloride, potassium iodide, and combinations thereof.

15. The method of claim 1, wherein the organic solvent is selected from methanol, ethanol, propanol, isopropanol, n-butanol, t-butanol, heptane, toluene, n-octane, and combinations thereof.

16. The method of claim 1, wherein the mixture is substantially free of organic solvents.

17. The method of claim 1, wherein the compound of Formula I is selected from 2-chloropyridine, 2,3-dichloropyridine, 2,6-dichloropyridine, and combinations thereof.

18. The method of claim 1, wherein the compound of Formula II is selected from 2-hydrazinopyridine, (3-chloro-2-pyridyl)hydrazine, (6-chloro-2-pyridyl)hydrazine, and combinations thereof.

19. The method of claim 1, wherein the method step of reacting the mixture occurs at a temperature in the range of about 90° C. to about 115° C.

20. The method of claim 1, wherein the method step of reacting the mixture occurs during a reaction time in the range of about 6 hours to about 30 hours.

21. The method of claim 1, wherein the method step of reacting the mixture occurs at a pressure in the range of about 1.0332 kg/cm² to about 5 kg/cm².
